(19) United States
(12) Patent Application Publication
(10) Pub. No.: US 2025/0114022 A1
(43) Pub. Date: Apr. 10, 2025

(54) DRIVING ASSISTANCE DEVICE, DRIVING ASSISTANCE METHOD, AND STORAGE MEDIUM

(71) Applicant: HONDA MOTOR CO., LTD., Tokyo (JP)

(72) Inventors: Naotoshi Fujimoto, Wako-shi (JP); Ryo Kimura, Wako-shi (JP); Yuya Kishimoto, Wako-shi (JP); Shigenobu Mitsuzawa, Wako-shi (JP); Satoru Shinkawa, Wako-shi (JP); Nozomi Nagatani, Wako-shi (JP); Junichi Sakamoto, Wako-shi (JP)

(21) Appl. No.: 18/904,181

(22) Filed: Oct. 2, 2024

(30) Foreign Application Priority Data
Oct. 6, 2023 (JP) 2023-174079

(51) Int. Cl. A61B 5/18; A61B 5/00; B60K 35/28

(52) U.S. Cl. CPC A61B 5/18; A61B 5/7264; B60K 35/28; B60K 2360/16; B60K 2360/741

(57) ABSTRACT

A plurality of questions for acquiring a plurality of pieces of driver information related to life of a driver of a moving object are output, the plurality of pieces of driver information are acquired on the basis of answers of the driver to the plurality of respective questions, a driver state related to at least one of a psychological state and a physical state of the driver is classified on the basis of the plurality of pieces of driver information, assistance information including advice regarding driving is generated on the basis of a classification result of the driver state, and the assistance information is output.

### INCORPORATION BY REFERENCE

The present application claims priority under 35 U.S.C. § 119 to Japanese Patent Application No. 2023-174079 filed on Oct. 6, 2023. The content of the application is incorporated herein by reference in its entirety.

### BACKGROUND

### Technical Field

The present invention relates to a driving assistance device, a driving assistance method, and a storage medium.

### Related Art

In recent years, efforts to provide access to sustainable transportation systems in consideration of vulnerable people among traffic participants have been gaining momentum. In order to realize this, research and development for further improving traffic safety and convenience is focused on research and development regarding driving assistance techniques. For example, WO2021/014632 A discloses a technique of acquiring biological information of a driver of a vehicle by using a biological sensor, classifying a state of the driver by analyzing the biological information, and providing information to the driver according to a classification result.

### SUMMARY

In a driving assistance technique, it is desirable to quickly estimate or classify a state of a driver. Therefore, it has been desired to quickly obtain information regarding a driver with a simpler configuration than a conventional method that requires a complicated sensor or the like.

In order to solve the above problems, an object of the present application is to realize a technique through which information regarding a state of a driver who drives a moving object can be quickly obtained with a simple configuration. Such an object contributes to development of a sustainable transportation system.

According to one aspect of the present invention, there is provided is a driving assistance device that executes: outputting a plurality of questions for acquiring a plurality of pieces of driver information related to a life of a driver of a moving object; acquiring the plurality of pieces of driver information on the basis of answers of the driver to the plurality of respective questions; classifying a driver state related to at least one of a psychological state and a physical state of the driver on the basis of the plurality of pieces of driver information; generating assistance information including advice regarding driving on the basis of a classification result of the driver state; and outputting the assistance information.

According to one aspect of the present invention, information regarding a state of a driver who drives a moving object can be quickly obtained with a simple configuration. Thus, it is possible to quickly classify the state of the driver and assist the driver, which can contribute to the development of a sustainable transportation system.

### BRIEF DESCRIPTION OF DRAWINGS

FIG. 1 is a diagram illustrating a configuration example of a system including a mobile terminal according to a first embodiment;

FIG. 2 is a block diagram illustrating a configuration example of a main part of a system;

FIG. 3 is a diagram illustrating a configuration example of driver information;

FIG. 4 is a diagram illustrating an example of display setting data;

FIG. 5 is a flowchart illustrating an operation example of a mobile terminal;

FIG. 6 is a flowchart illustrating an operation example of the mobile terminal;

FIG. 7 is a diagram illustrating a display example in the mobile terminal;

FIG. 8 is a diagram illustrating an example of display setting data in a second embodiment;

FIG. 9 is a diagram illustrating an example of display setting data in the second embodiment; and

FIG. 10 is a flowchart illustrating an operation example of the mobile terminal in the second embodiment.

### DETAILED DESCRIPTION

### 1. First Embodiment

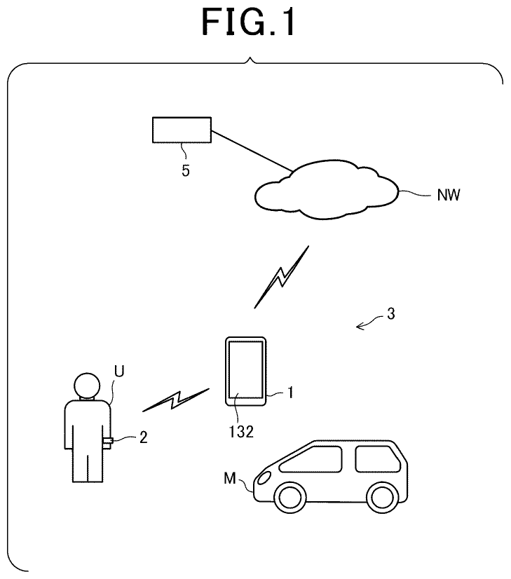

A first embodiment to which the present invention is applied will be described with reference to FIGS. 1 to 7.

### 1-1. Configuration of System Including Mobile Terminal

FIG. 1 is a diagram illustrating a configuration example of a system including a mobile terminal 1.

The mobile terminal 1 is a device used by a user U who drives a vehicle M. The user U is a driver of the vehicle M, and can use the mobile terminal 1 before getting on the vehicle M. The mobile terminal 1 is, for example, a portable computer, and is specifically any of a smartphone, a tablet computer, a laptop computer, and other computers.

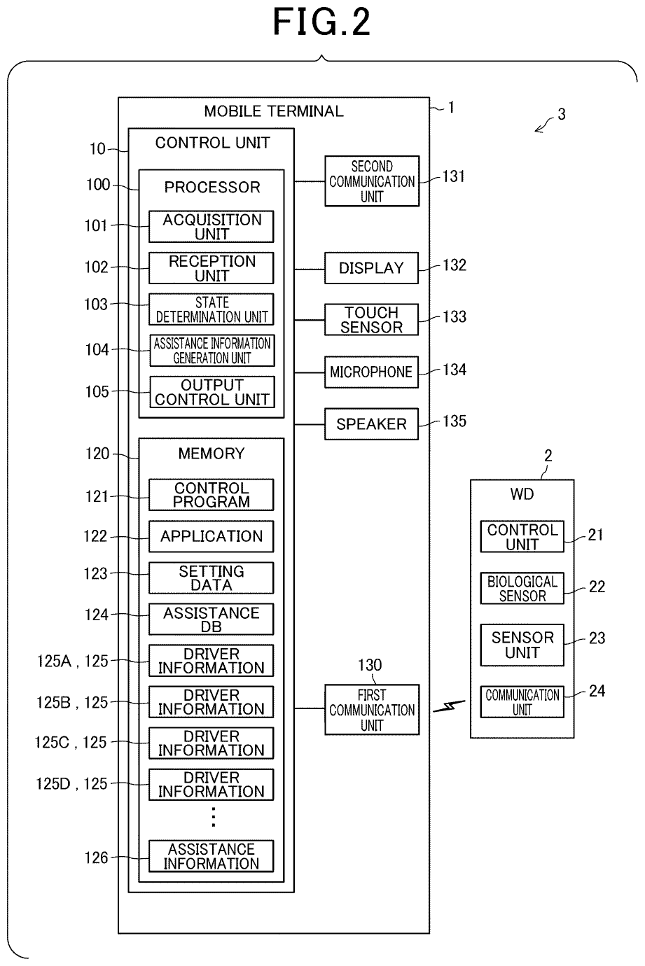

The mobile terminal 1 includes a display 132 and a speaker 135 (FIG. 2) that will be described later. The mobile terminal 1 may output information to the user U by displaying an image or text on the display 132 and outputting a voice from the speaker 135.

The mobile terminal 1 outputs a question to the user U before the user U drives the vehicle M. For example, the mobile terminal 1 outputs a question before the user U gets on the vehicle M. The content of the question is a question for obtaining information related to the life of the user U, and will be specifically described later. In a case where the user U inputs an answer to the question, the mobile terminal 1 estimates and classifies a state of the user U on the basis of the answer of the user U. The mobile terminal 1 generates assistance information on the basis of the classified state of the user U. The assistance information is information that assists the user U in driving the vehicle M. The mobile terminal 1 outputs the assistance information from at least one of the display 132 and the speaker 135 to assist the user U in driving the vehicle M.

The mobile terminal 1 performs wireless communication with a wearable device (hereinafter, referred to as a WD) 2 used by the user U. The mobile terminal 1 configures a communication system 3 together with the WD 2.

The WD 2 is a device worn on the body or clothes of the user U. The WD 2 is, for example, an electronic device having a wristwatch shape, a dongle shape, a glasses shape, or another shape. The WD 2 executes detection related to the user U by using various sensors that will be described later. For example, the WD 2 detects biological information of the user U and a body motion of the user U. Examples of the biological information of the user U include a pulse, a blood pressure, a respiratory rate, a blood glucose level, a perspiration state, and other vital data. The WD 2 generates information related to the life of the user U on the basis of detection results from the sensors. The information related to the life of the user U is, for example, a sleeping time of the user U, a walking amount of the user U, an activity amount of the user U, a tense state of the user U, and various types of information called a so-called life log. The information generated by the WD 2 may be collectively referred to as information regarding a life behavior of the user U. The WD 2 executes communication with the mobile terminal 1 and transmits the information regarding the life behavior of the user U to the mobile terminal 1.

In a case where the mobile terminal 1 has acquired the information regarding the life behavior of the user U from the WD 2, the mobile terminal 1 can estimate and classify the state of the user U on the basis of the information acquired from the WD 2.

As illustrated in FIG. 1, the mobile terminal 1 may be configured to be able to communicate with a server 5 via a communication network NW.

The communication network NW is a communication network including a public network, a dedicated line, other communication circuits, and the like. The server 5 is a computer that transmits and receives data to and from various devices via the communication network NW. The server 5 may be a single server computer, a plurality of server computers, or a cloud server. For example, the mobile terminal 1 executes data communication with the server 5 as necessary in the process of generating the assistance information.

### 1-2. Configuration of Mobile Terminal

FIG. 2 is a block diagram illustrating a configuration example of the communication system 3, and illustrates configurations of the mobile terminal 1 and the WD 2.

The mobile terminal 1 includes a control unit 10, and includes a first communication unit 130, a second communication unit 131, a display 132, a touch sensor 133, a microphone 134, and a speaker 135 controlled by the control unit 10.

The first communication unit 130 and the second communication unit 131 are wireless communication devices including a transmitter that transmits data and a receiver that receives data. The first communication unit 130 executes wireless data communication with a device different from the mobile terminal 1 by using Bluetooth (registered trademark), Wi-Fi (registered trademark), or another short-range wireless communication method. For example, the first communication unit 130 executes communication with the WD 2. In this case, the WD 2 is an example of another device.

The second communication unit 131 executes wireless data communication by using a communication method different from that of the first communication unit 130. The second communication unit 131 is connected to the communication network NW through cellular communication, for example, and executes data communication with the server 5 via the communication network NW. The first communication unit 130 is an example of a communication unit.

The display 132 has a display screen including a liquid crystal display panel or an organic electroluminescence (EL) panel. The display 132 displays various screens including images and text under the control of the control unit 10.

The touch sensor 133 is a sensor that detects a touch operation of the user U. In the present embodiment, the touch sensor 133 is disposed to overlap the display 132 and is integrated with the display 132 to configure a touch panel. The touch sensor 133 detects an operation of the user U touching a surface of the display 132, and outputs operation data indicating a position of the detected operation to the control unit 10.

The microphone 134 detects a sound to generate an audio signal, converts the generated audio signal into digital audio data, and outputs the digital audio data to the control unit 10. For example, the control unit 10 can detect a voice of the user U with the microphone 134.

The speaker 135 outputs sound on the basis of an audio signal or digital audio data input from the control unit 10.

The display 132 is an example of a display unit. The display 132 and the speaker 135 function as an output unit for the mobile terminal 1 to perform output. The touch sensor 133 and the microphone 134 function as an input unit through which the mobile terminal 1 receives an input from the user U.

The control unit 10 includes a processor 100 and a memory 120. The processor 100 is a computer including a central processing unit (CPU), a micro processing unit (MPU), or another integrated circuit. The memory 120 is a storage device that stores programs and data. The processor 100 may use a volatile random access memory (RAM) as a work area. The RAM may be integrated and mounted in the processor 100, or the memory 120 may include the RAM.

The memory 120 is a rewritable nonvolatile storage device, and stores a program executed by the processor 100 and data processed by the processor 100. The memory 120 includes, for example, a semiconductor storage device such as a flash read only memory (ROM) or a solid state disk (SSD), or a magnetic storage device. The memory 120 stores a control program 121, an application program (hereinafter, referred to as an application) 122, setting data 123, an assistance database (hereinafter, referred to as a DB) 124, driver information 125A, 125B, 125C, and 125D, and assistance information 126.

The control program 121 and the application 122 are programs executed by the processor 100, and are stored in the memory 120 to be readable by the processor 100. The control program 121 is a basic control program for the processor 100 to control each unit of the mobile terminal 1, and is an operating system (OS). The application 122 is an application program executed on the OS.

The setting data 123 is data that is referred to in a case where the processor 100 executes the application 122. The setting data 123 includes a setting value or the like related to the function of the application 122.

The assistance DB 124 is a database that stores image data and the like used by an assistance information generation unit 104 that will be described later.

The driver information 125 is information acquired by an acquisition unit 101.

The assistance information 126 is information generated by the assistance information generation unit 104.

The processor 100 includes, as functional units, the acquisition unit 101, a reception unit 102, a state determination unit 103, the assistance information generation unit 104, and an output control unit 105. These functional units are realized by the processor 100 executing the application 122. The application 122 is an example of a program.

The acquisition unit 101 acquires information related to the life of the user U. The reception unit 102 receives an input from the user U. Specifically, in a case where the touch sensor 133 detects a touch operation, the reception unit 102 specifies input content based on the touch operation and generates input data indicating the specified content. The reception unit 102 analyzes the voice of the user U detected by the microphone 134 and executes a text conversion process, thereby generating input data indicating the content uttered by the user U.

The acquisition unit 101 outputs a question to the user U by using the display of the display 132 and the output of the voice from the speaker 135. The acquisition unit 101 acquires an answer to the question on the basis of the input data received by the reception unit 102 after outputting the question, and generates the driver information 125A, 125B, 125C, and 125D on the basis of the acquired answer. Hereinafter, the driver information 125A, 125B, 125C, and 125D will be referred to as driver information 125 in a case where the driver information is not distinguished. In the present embodiment, an example in which the mobile terminal 1 acquires the four pieces of driver information 125A, 125B, 125C, and 125D by the acquisition unit 101 will be described, but the number of pieces of driver information 125 acquired by the acquisition unit 101 and the number of pieces of driver information 125 stored in the memory 120 are not limited.

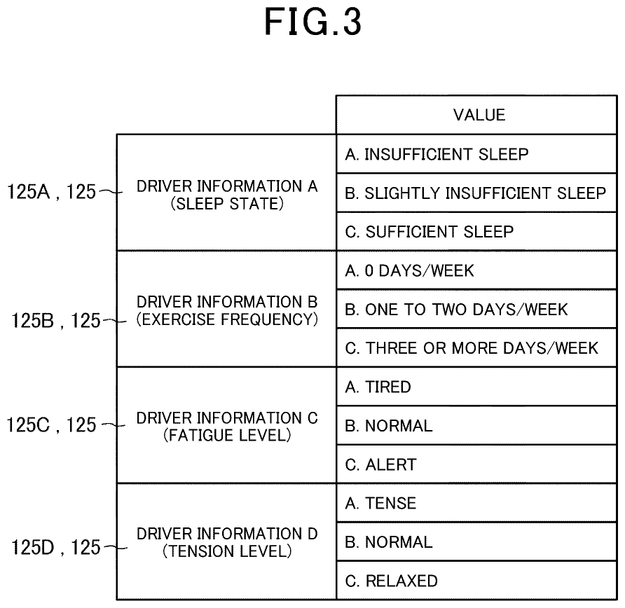

FIG. 3 is a diagram illustrating a configuration example of the driver information 125.

In the present embodiment, the driver information 125A is information related to a sleep state of the user U, and the driver information 125B is information related to an exercise frequency of the user U. The driver information 125C is information related to a fatigue level of the user U, and the driver information 125D is information related to a tension level of the user U. The driver information 125A, 125B, 125C, and 125D may be driver information A, B, C, and D, respectively.

There is no limitation on values that can be taken by the driver information 125A to 125D. In the present embodiment, each piece of the driver information 125A to 125D has one of values A, B, and C.

Specifically, the driver information 125A is information regarding recent sleep of the user U with reference to the date on which A is generated as a value of the driver information 125. The value A of the driver information 125A indicates that the user U does not have sufficient sleep, and the value B indicates that sleep of the user U is slightly insufficient. The value C of the driver information 125A indicates that the user U has sufficient sleep, that is, that the user U sleeps well.

Specifically, the driver information 125B is information regarding the number of days of exercise in one week, and exercise indicates exercise with an amount of exercise equivalent to walking for 20 minutes or more, or an amount of exercise equal to or more than that, for example. The value A of the driver information 125B indicates that there is no number of days of exercise, that is, the number of days of exercise is 0 per week. The value B of the driver information 125B indicates that the number of days of exercise is one or two per week, and the value C indicates that the number of days of exercise is three or more per week.

Specifically, the driver information 125C is information regarding the degree of fatigue of the user U at the time of generating the driver information 125C. The value A of the driver information 125C indicates that the user U is tired, and the value C indicates that the user U is alert. The value B of the driver information 125C indicates that the user U is normal, that is, the state of the user U does not correspond to either a tired state or an alert state.

The driver information 125D is, specifically, information regarding whether the user U is tense or relaxed at the time of generating the driver information 125D. The value A of the driver information 125D indicates that the user U is tense, and the value C indicates that the user U is relaxed. The value B of the driver information 125D indicates that the user U is normal, that is, the state of the user U does not correspond to either a tense state or a relaxed state.

The acquisition unit 101 outputs a question for determining each value of the driver information 125A to 125D. For example, the acquisition unit 101 outputs a question for determining whether the value of the driver information 125A is A, B, or C. The same applies to the driver information 125B to 125D.

The acquisition unit 101 determines the value of each of the plurality of pieces of driver information 125A to 125D included in the driver information 125 by analyzing the input content received by the reception unit 102 after outputting the question. Determination of the value of the driver information 125 corresponds to generation of the driver information 125.

In a case where data communication with the WD 2 can be performed by the first communication unit 130, the acquisition unit 101 receives and acquires information regarding a life behavior from the WD 2. In a case where the information acquired from the WD 2 corresponds to any of the driver information 125A to 125D, the acquisition unit 101 determines a value of the driver information 125 on the basis of the acquired information. The acquisition unit 101 omits outputting of a question for the driver information 125 of which a value is determined on the basis of the information acquired from the WD 2. For example, in a case of determining a value of the driver information 125A on the basis of the information acquired from the WD 2, the acquisition unit 101 does not output a question corresponding to the driver information 125A. In a case of determining values of all pieces of the driver information 125 on the basis of the information acquired from the WD 2, the acquisition unit 101 does not output a question to the user U.

The state determination unit 103 classifies a driver state related to the state of the user U on the basis of the driver information 125. The driver state is a state related to at least one of a psychological state and a physical state of the user U. For example, the driver state is an index reflecting an attention or a cognitive function of the user U, a perception ability of the user U, an operation ability of the user U, a judgment ability of the user U, and the like. The driver state may be a plurality of index values reflecting the attention or the cognitive function of the user U, the perception ability of the user U, the operation ability of the user U, the judgment ability of the user U, and the like, or may be one index value obtained by integrating these. An index value of the driver state may be expressed in a plurality of preset stages or may be expressed by a numerical value. According to the present embodiment, in the state determination unit 103, the driver state of the user U is set to an index value indicating any of a positive state, a negative state, or an intermediate state between the positive state and the negative state. That is, state determination unit 103 expresses the driver state by three stages of indices. This is an example, and for example, the state determination unit 103 may classify the driver state into four or more stages including a positive state, a negative state, and an intermediate state. Specifically, the state determination unit 103 may classify the driver state into five stages of a positive state, a negative state, an intermediate state between the positive state and the negative state, a quasi-positive state between the positive state and the intermediate state, and a quasi-negative state between the intermediate state and the negative state, or may classify the driver state into other four or more stages.

The state determination unit 103 can estimate a change in the driver state. For example, a time point at which the driver information 125 stored in the memory 120 is generated or a time point at which an answer for generating the driver information 125 is received will be referred to as a first time point, and a time point after a predetermined time has elapsed from the first time point will be referred to as a second time point. As described above, the state determination unit 103 classifies the driver state reflecting the state of the user U at the first time point. The state determination unit 103 may estimate the state of the user U at the second time point and classify the estimated driver state. A length of the predetermined time is not limited. For example, the predetermined time may be set within one hour such that the user U continues driving the vehicle M at the second time point. The predetermined time may be about 6 hours to 12 hours, and in this case, a change in the physical condition of the user U in one day can be estimated. Among the driver states classified by the state determination unit 103, the driver state at the first time point will be referred to as a first driver state, and the driver state at the second time point will be referred to as a second driver state.

As an example, it is assumed that the values of the driver information 125A and 125B are both A, the value of the driver information 125C is B, and the value of the driver information 125D is C. In this example, the user U is in a physically fatigued state, but is mentally relaxed. Thus, for example, the state determination unit 103 classifies the first driver state as an intermediate state. In this example, the user U is in an intermediate state in a case of starting driving of the vehicle M, but since it is assumed that fatigue accumulates during driving, the state determination unit 103 classifies the second driver state as a negative state. In this case, the driver state of the user U deteriorates from the intermediate state to the negative state.

As another example, it is assumed that the values of the driver information 125A and 125B are both C, the value of the driver information 125C is B, and the value of the driver information 125D is A. In this example, the user U is in a state of less physical fatigue, but is mentally tense. Thus, for example, the state determination unit 103 classifies the first driver state as an intermediate state. In this example, the user U is in the intermediate state in a case of starting driving the vehicle M, but it is assumed that the tension is released and the user U relaxes while driving the vehicle M. Therefore, the state determination unit 103 classifies the second driver state as a positive state. In this case, the driver state of the user U is improved from the intermediate state to the positive state.

A method in which the state determination unit 103 estimates and classifies the first driver state and the second driver state is not limited. For example, a table in which a combination of values of the driver information 125A to 125D, a classification of the first driver state, and a classification of the second driver state are associated with each other may be used. This table may be stored in advance in the memory 120 as the setting data 123, for example.

For example, the state determination unit 103 may obtain the driver state from the driver information 125 by using a function or a program. That is, the mobile terminal 1 has a function or a program for obtaining a classification of the first driver state from the combination of the values of the driver information 125A to 125D. Similarly, the mobile terminal 1 has a function or a program for obtaining a classification of the second driver state from the combination of the values of the driver information 125A to 125D. These functions or programs may be stored in the memory 120 in advance as the setting data 123, for example.

For example, the state determination unit 103 may obtain the driver state by calculating a value of the driver information 125. That is, the mobile terminal 1 replaces each value of the driver information 125 with a calculable score such as a numerical value, and sums scores of the plurality of pieces of driver information 125. The mobile terminal 1 converts a total value of the scores into a driver state. In this configuration, data for associating the score with the driver state is stored in advance in the memory 120 as the setting data 123, for example.

For example, the state determination unit 103 may have a learning model that associates the value of the driver information 125 with the driver state. That is, the mobile terminal 1 may have a trained artificial intelligence (AI) model that has learned a correlation between the value of the driver information 125 and the classification of the driver state. This trained model has learned a correlation between the combination of the values of the driver information 125A to 125D and the classification of the first driver state and a correlation between the combination of the values of the driver information 125A to 125D and the classification of the second driver state. In a case where a predetermined time is designated by the trained model, the second driver state after the designated time has elapsed may be estimated. This trained model or training data may be stored in the memory 120 in advance as the setting data 123, for example.

The above-described various examples are examples in which the state determination unit 103 individually classifies the first driver state and the second driver state. This is an example, and for example, the state determination unit 103 may collectively classify the first driver state and the second driver state. Specifically, the state determination unit 103 may estimate a combination of the classification of the first driver state and the classification of the second driver state. For example, the state determination unit 103 may be configured to classify one index obtained by integrating the first driver state and the second driver state, and in this case, the state determination unit 103 does not perform a process of individually classifying the first driver state and the second driver state.

The assistance information generation unit 104 generates the assistance information 126 on the basis of the result of classification of the driver state from the state determination unit 103, and stores the assistance information 126 in the memory 120.

The assistance information 126 is information output from the mobile terminal 1 to the user U. The assistance information 126 may include a still image, a moving image, a voice, or other content, and in the present embodiment, the assistance information 126 including a still image and a voice is exemplified.

The assistance information 126 includes a message including text. This message is output by being displayed or in a voice. This message is information for assisting the user U in driving the vehicle M, and can be regarded as advice. Specifically, this message is a matter to be noted when the user U drives the vehicle M. More specifically, the advice included in the assistance information 126 is attention point advice in cognition, judgment, and operation at the time of driving.

The assistance information 126 includes a still image or a moving image to be displayed on the display 132. The image included in the assistance information 126 expresses at least one of a driver state and information for advising the user U to drive the vehicle M.

An aspect of the output of the assistance information 126 and an information amount of the assistance information 126 are devised to reduce the cognitive load of the user U. Specifically, the assistance information 126 is configured to be suitable for the user U to recognize and store the content of the assistance information 126.

In order to output the assistance information 126 in an aspect in which the assistance information 126 easily remain in the memory of the user U, the number of pieces of information displayed on the display 132 is preferably four or less, and more preferably two or less. The number of pieces of information output in a voice from the speaker 135 is also preferably four or less, and more preferably two or less.

In the present embodiment, in order to reduce an information amount of the assistance information 126 displayed on the display 132, the assistance information 126 including an image imitating a weather forecast is generated. The weather forecast is suitable for allowing a human to recognize both a state at a certain time point and a state after a lapse of time and changes in the states. Many people have social common recognition for weather forecast. Specifically, it can be considered that the user U generally recognizes that the weather forecast indicates an outline of an event by using an image, and that the certainty of the weather forecast is limited. Since the weather is a matter of concern to many people, the weather forecast attracts attention of many people.

In a case where the driver state is indicated by an image imitating the weather forecast, it can be expected that the user U pays attention to this image, or even if a physical state that the user U is aware of is different from the driver state, the difference is allowed. Since many people are familiar with the image of the weather forecast, there is an advantage that the user U can quickly understand his/her state since the driver state is indicated by the image imitating the weather forecast. In a case where four or less images or two or less images are displayed on the display 132 as such images, there is an effect that the user U can easily remember the images during driving of the vehicle M and easily recall the images during driving. Therefore, appropriate advice regarding driving of the vehicle M can be given to the user U, and the user U can be effectively assisted in driving. If the message included in the assistance information 126 is a comment related to the image imitating the weather forecast, advice that is more likely to remain in the awareness of the user U can be given.

The assistance DB 124 stores messages and images used for generating the assistance information 126. The assistance information generation unit 104 acquires messages and images corresponding to the first driver state and the second driver state from the assistance DB 124. A storage form of information in the assistance DB 124 is not limited. For example, the assistance DB 124 stores messages and images in association with a combination of the first driver state and the second driver state.

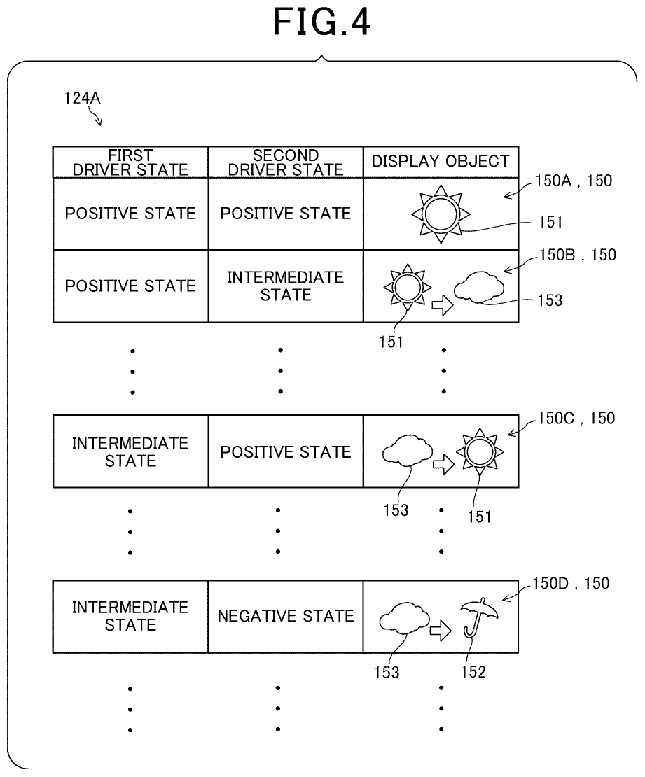

FIG. 4 is a diagram illustrating an example of the display setting data 124A. The display setting data 124A is data stored in the assistance DB 124, and includes a display object 150 in association with the first driver state and the second driver state.

In the example in FIG. 4, the display setting data 124A associates the combination of the first driver state and the second driver state with the display object 150. FIG. 4 illustrates display objects 150A, 150B, 150C, and 150D, but the number of display objects 150 is not limited. For example, the display setting data 124A associates one display object 150 with one combination of the first driver state and the second driver state. Hereinafter, the display objects 150A, 150B, 150C, 150D, and the like will be referred to as a display object 150 in a case where the display objects not distinguished from one another.

As described above, the display object 150 according to the present embodiment is a weather forecast metaphor. The display object 150 is a set of images including a first object 151 representing sunny weather, a second object 152 representing rainy weather, and a third object 153 representing cloudy weather. For example, in the display setting data 124A, a combination in which the first driver state is a positive state and the second driver state is a positive state is associated with the display object 150A. The display object 150A includes the first object 151 representing sunny, and indicates that the driver state of the user U maintains a good state.

For example, in the display setting data 124A, a combination in which the first driver state is a positive state and the second driver state is an intermediate state is associated with the display object 150B. The display object 150B includes a combination of the first object 151 and the third object 153, and indicates “sunny and occasionally cloudy” weather. That is, the display object 150B indicates that there is a probability that the driver state of the user U is initially good and is lowered thereafter.

The mobile terminal 1 of the present embodiment expresses the driver state of the user U by using, for example, metaphors of seven types of weather including bright and clear, sunny, sunny and then cloudy, cloudy and then sunny, cloudy, cloudy and then rainy, rainy and then cloudy, and rainy. That is, the display setting data 124A associates the display objects 150 indicating the seven types of weather with the driver state.

The display setting data 124A may be in a table form including the image data of the display object 150 as illustrated in FIG. 4, or may be a list or a function including data designating the image data of the display object 150. In this case, the image data of the display object 150 may be stored in the assistance DB 124 separately from the display setting data 124A.

The assistance information generation unit 104 acquires image data of the display object 150 corresponding to the driver state on the basis of the display setting data 124A, and generates the assistance information 126 including the image data. In addition to the display object 150, the assistance information generation unit 104 may cause a display object including an image of an icon or the like to be included in the assistance information 126,

The output control unit 105 outputs the assistance information 126. The output control unit 105 outputs, from the speaker 135, the text included in the assistance information 126 in a voice, and displays, on the display 132, the display object 150 included in the assistance information 126. The assistance information 126 may include information for designating an output form. For example, the assistance information 126 may include information for designating an output form of the text included in the assistance information 126 as either output using a voice or display using the display 132. In this case, the output control unit 105 outputs the assistance information 126 in an output form designated by the assistance information 126.

The output control unit 105 preferably outputs the assistance information 126 before the user U drives the vehicle M. For example, the application 122 displays, on the display 132, a message or the like requesting the user U to operate the application 122 before driving the vehicle M. In a case of a configuration in which whether or not the mobile terminal 1 is located inside the vehicle M can be determined, the control unit 10 may be configured to be able to start the application 122 only while the mobile terminal 1 is located outside the vehicle M. This configuration may be realized as a configuration in which the mobile terminal 1 can communicate with an in-vehicle device (not illustrated) mounted on the vehicle M through near field communication, and determines whether or not the mobile terminal 1 is located inside the vehicle M by detecting a communication state between the in-vehicle device and the mobile terminal 1.

### 1-3. Configuration of Wearable Device

The WD 2 includes a control unit 21, and a biological sensor 22, a sensor unit 23, and a communication unit 24 controlled by the control unit 21.

The control unit 21 includes a processor and a memory (not illustrated). The processor configuring the control unit 21 is a computer including a CPU, an MPU, or another integrated circuit. The memory is a storage device that stores a program executed by the processor and data processed by the processor. The control unit 21 may be an integrated circuit (IC) in which a processor, a memory, and other peripheral circuits are integrated.

The biological sensor 22 is a sensor that detects biological information of the user U. The biological sensor 22 detects, for example, a pulse, a blood pressure, a blood oxygen concentration, a respiratory rate, a blood glucose level, a perspiration state, and other vital data. Specifically, the biological sensor 22 is a heart rate sensor, and may be a blood pressure sensor, a blood oxygen concentration sensor, a perspiration sensor, or the like. The sensor unit 23 is a unit in which a sensor that detects the movement of the WD 2 is integrated. The sensor unit 23 includes an acceleration sensor, a rotational angular acceleration sensor or a gyro sensor, a magnetic field sensor, and the like. The sensor unit 23 may include a sensor that measures an external environment, and specifically may include an atmospheric pressure sensor or a temperature sensor. The sensor unit 23 may be an inertial measurement unit (IMU) in which these sensors are integrated into one package.

The communication unit 24 is a wireless communication device including a transmitter that transmits data and a receiver that receives data. The communication unit 24 executes wireless data communication with a device different from the WD 2 by using Bluetooth (registered trademark), Wi-Fi (registered trademark), or another short-range wireless communication method. For example, the communication unit 24 executes communication with the mobile terminal 1.

The WD 2 may include a display unit including a liquid crystal display panel, an organic EL panel, or a light emitting diode (LED). The WD 2 may include an input unit such as a switch or a touch sensor.

The control unit 21 generates the information regarding the life behavior of the user U as described above on the basis of at least one of a detection result from the biological sensor 22 and a detection result from the sensor unit 23. For example, the control unit 21 generates information such as a sleeping time of the user U, a walking amount of the user U, an activity amount of the user U, a fatigue level of the user U, and a tense state of the user U.

The control unit 21 is connected to the mobile terminal 1 through wireless communication by the communication unit 24, and transmits the information regarding the life behavior of the user U to the mobile terminal 1 in a case where information transmission is requested from the mobile terminal 1. The control unit 21 may transmit the detection result from the biological sensor 22 and the detection result from the sensor unit 23 in response to a request from the mobile terminal 1.

### 1-4. Operations of Mobile Terminal

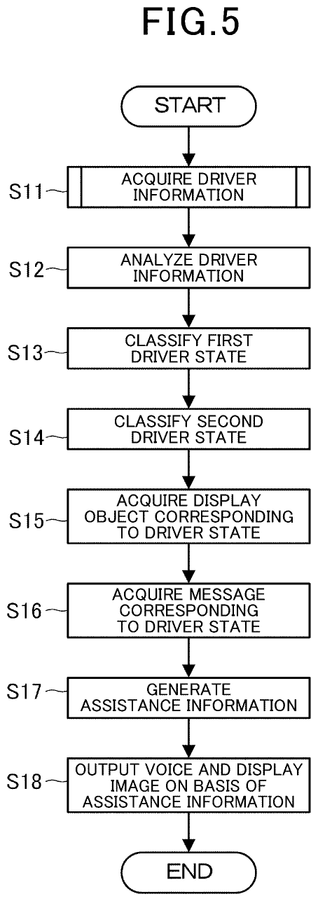

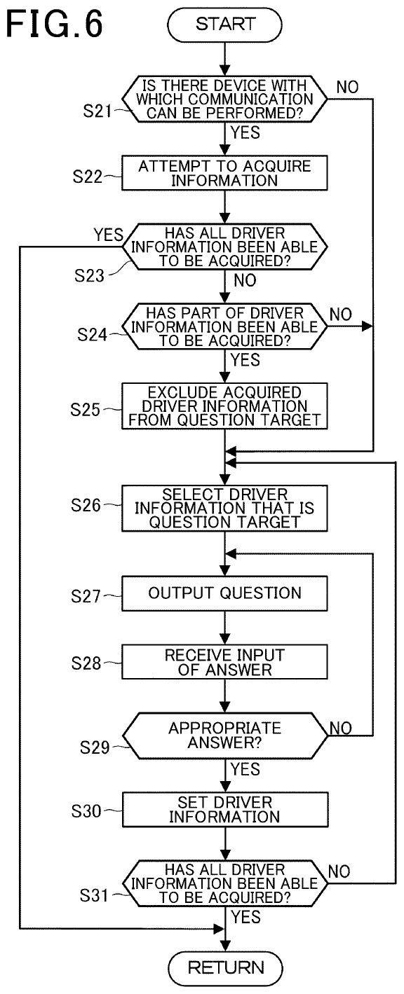

FIGS. 5 and 6 are flowcharts illustrating an operation example of the mobile terminal 1. FIG. 6 illustrates step S11 in FIG. 5 in detail.

In the operations in FIG. 5, steps S12 to S14 are executed by the state determination unit 103, steps S15 to S17 are executed by the assistance information generation unit 104, and step S18 is executed by the output control unit 105. In the operations in FIG. 6 corresponding to step S11, steps S21 to S27 and S29 to S31 are executed by the acquisition unit 101, and step S28 is executed by the reception unit 102.

The mobile terminal 1 acquires the driver information 125 (step S11).

Specifically, as illustrated in FIG. 6, the mobile terminal 1 determines the presence or absence of a device that can perform communication with the first communication unit 130 (step S21). In a case where there is no device that can perform communication with the first communication unit 130 (step S21: NO), the mobile terminal 1 proceeds to step S26 that will be described later.

In a case where there is a device that can perform communication with the first communication unit 130 (step S21: YES), the mobile terminal 1 attempts to acquire information from this device (step S22). For example, the mobile terminal 1 executes communication with the WD 2 in step S22, requests the WD 2 for information regarding a life behavior of the user U, and acquires information transmitted by the WD 2 in response to the request.

The mobile terminal 1 determines whether or not all the driver information 125 has been able to be acquired through the operation in step S22 (step S23). In step S23, the mobile terminal 1 determines that the driver information 125 has been able to be acquired in a case where the information has been successfully received by the first communication unit 130 and the received information is information that can be used as the driver information 125. In a case where all the necessary driver information 125 has been able to be acquired (step S23: YES), the mobile terminal 1 skips the operations in steps S24 to S31 and proceeds to step S12 in FIG. 5.

In a case where the driver information 125 acquired in step S22 is a part of the necessary driver information 125 and in a case where the driver information 125 has been unable to be acquired, the mobile terminal 1 makes a negative determination in step S23 (step S23: NO). In this case, the mobile terminal 1 determines whether or not a part of the necessary driver information 125 has been able to be acquired in step S22 (step S24).

In step S22, in a case where the driver information 125 has been unable to be acquired (step S24: NO), the mobile terminal 1 proceeds to step S26 that will be described later.

In a case where a part of the necessary driver information 125 has been able to be acquired in step S22 (step S24: YES), the mobile terminal 1 excludes the acquired driver information 125 from a question target (step S25), and then proceeds to step S26.

In step S26, the mobile terminal 1 selects the driver information 125 that is a question target (step S26). The mobile terminal 1 outputs a question for acquiring the selected driver information 125 (step S27). Subsequently, the mobile terminal 1 outputs the question by using, for example, at least one of display using the display 132 or a voice output from the speaker 135 (step S27).

The mobile terminal 1 receives an input of an answer to the question (step S28). The mobile terminal 1 determines whether or not the answer received in step S28 is an appropriate answer (step S29). The appropriate answer is an answer that allows one of values A, B, and C to be determined for the driver information 125 selected in step S26.

In a case where the answer received in step S28 is not an appropriate answer (step S29: NO), the mobile terminal 1 returns to step S27. In a case where the answer received in step S28 is an appropriate answer (step S29: YES), the mobile terminal 1 sets the value of the driver information 125 determined on the basis of the answer (step S30). As a result, the driver information 125 selected in step S26 by the mobile terminal 1 has been acquired.

The mobile terminal 1 determines whether all the driver information 125 has been able to be acquired (step S31). All the driver information 125 in step S31 is the driver information 125 to be acquired by asking a question at the time of starting step S26. In a case where there is unacquired driver information 125 (step S31: NO), the mobile terminal 1 returns to step S26. In a case where all the driver information 125 has been able to be acquired (step S31: YES), the mobile terminal 1 proceeds to step S12 in FIG. 5.

The mobile terminal 1 analyzes the driver information 125 acquired in step S11 (step S12), classifies the first driver state (step S13), and classifies the second driver state (step S14).

The mobile terminal 1 acquires the display object 150 corresponding to the first driver state and the second driver state from the assistance DB 124 (step S15). The mobile terminal 1 acquires a message corresponding to the first driver state and the second driver state from the assistance DB 124 (step S16).

The mobile terminal 1 generates the assistance information 126 including the display object 150 and the message acquired in step S15 and step S16 (step S17). The mobile terminal 1 executes voice output from the speaker 135 and display using the display 132 on the basis of the assistance information 126 (step S18).

The assistance DB 124 may store a message associated with each of the display objects 150. In this case, the state determination unit 103 acquires a message corresponding to the display object 150 from the assistance DB 124 in step S16. Alternatively, a message may be stored in the assistance DB 124 in association with a combination of values of the driver information 125. In this case, the state determination unit 103 acquires a message corresponding to a combination of values of the driver information 125 from the assistance DB 124 in step S16.

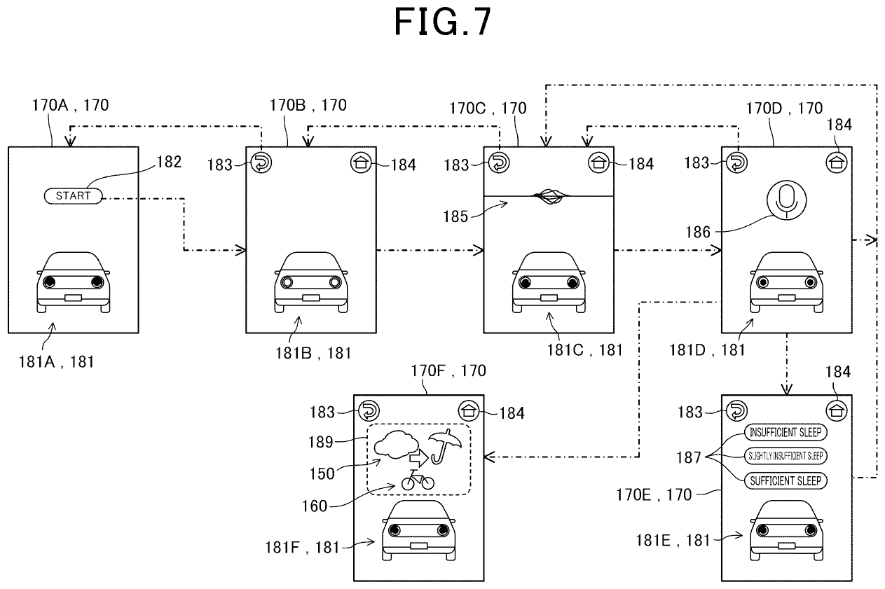

FIG. 7 is a diagram illustrating a display example in the mobile terminal 1. FIG. 7 illustrates transitions of screens 170A, 170B, 170C, 170D, 170E, and 170F configuring a screen 170 displayed on the display 132 as an example of the screen 170. Hereinafter, the screens 170A to 170F will be referred to as a screen 170 in a case where the screens are not distinguished from each other.

After the application 122 is started in the mobile terminal 1, the mobile terminal 1 displays the screen 170A while waiting for an operation of the user U. The screen 170A includes a vehicle object 181A. The vehicle object 181A is an image of a character displayed to have a conversation with the user U on the screen 170, and is, for example, an image imitating the vehicle M as illustrated in FIG. 7. The vehicle object 181A may be an icon that does not change, or may be a still image or an animation. The vehicle object 181A of the present embodiment is displayed as vehicle objects 181B to 181F having different expressions or impressions from the vehicle object 181A on the screens 170B to 170F. The vehicle objects 181A to 181F will be collectively referred to as a vehicle object 181. The vehicle object 181 is a character configuring an interactive user interface of the application 122, and has a role as an interaction partner of the user U.

Since the screen 170A is a screen displayed before the user U starts using the application 122, the vehicle object 181A is an image imitating a state in which the character is asleep.

The screen 170A includes a start instruction portion 182. The start instruction portion 182 is an image displayed on the display 132, and is an operation portion for the user U to give an instruction to start using the application 122. When detecting a touch operation on the start instruction portion 182, the mobile terminal 1 switches the display from the screen 170A to the screen 170B in response to the touch operation.

The screen 170B is displayed while the processor 100 prepares execution of the function of the application 122 in response to the operation on the start instruction portion 182. For example, the screen 170B is displayed while the processor 100 loads data regarding the application 122 or while initializing a work area of the memory 120. The vehicle object 181B disposed on the screen 170B is an image imitating a state in which a character wakes up.

The screen 170B includes a transition instruction portion 183 and a transition instruction portion 184. The transition instruction portions 183 and 184 are images displayed on the display 132. The transition instruction portion 183 is an operation portion for giving an instruction to return the display of the display 132 to the previous screen, and the transition instruction portion 184 is an operation portion for giving an instruction to transition to the screen immediately after the start of the application 122. For example, when the transition instruction portion 183 is operated while the screen 170B is displayed, the display of the display 132 returns to the screen 170A. For example, when the transition instruction portion 184 is operated on the screen 170B and the screens 170C to 170F that will be described later, the display of the display 132 returns to the screen 170A.

When preparation for execution of the function of the application 122 is completed during the screen 170B is displayed, the mobile terminal 1 switches the display of the display 132 from the screen 170B to the screen 170C, and outputs a voice of a question. This step corresponds to step S27 in FIG. 6. The screen 170C is displayed while the mobile terminal 1 outputs the voice of the question. On the screen 170C, a vehicle object 181C, transition instruction portions 183 and 184, and a busy display 185 are disposed. The busy display 185 is an indicator indicating that the mobile terminal 1 is outputting a voice. The busy display 185 may be an animation. The vehicle object 181C may be an image imitating a smile in order to recall a sense of closeness to the user U.

When the output of the voice of the question ends, the mobile terminal 1 switches the display of the display 132 from the screen 170C to the screen 170D. The screen 170D is displayed while input of an answer based on a voice or a touch operation is received. This step corresponds to step S28 in FIG. 6. On the screen 170D, a vehicle object 181D, transition instruction portions 183 and 184, and an utterance guide 186 are disposed. The utterance guide 186 is an image indicating that the mobile terminal 1 is in a state in which input based on a voice of the user U can be received. The utterance guide 186 may be an animation that changes in accordance with a detection state of a voice using the microphone 134. The vehicle object 181D may be an image imitating an expression different from that of the vehicle object 181C.

In a case where the mobile terminal 1 receives the voice of the user U and determines that the voice of the user U is not an appropriate answer, the mobile terminal 1 switches the display of the display 132 from the screen 170D to the screen 170E. That is, the mobile terminal 1 may display the screen 170E in a case where a negative determination is made in step S29 in FIG. 6. On the screen 170E, a vehicle object 181E, transition instruction portions 183 and 184, and an answer guide 187 are disposed. The vehicle object 181E is an image imitating a sad expression, for example, in order to notify that the user U has not made the required answer. The answer guide 187 is an image for prompting the user U to give an answer suitable for determining a value of the driver information 125. The answer guide 187 includes, for example, text that assists the user U in making an appropriate answer. The text of the answer guide 187 may be text indicating a value itself of the driver information 125. As illustrated in FIG. 7, the screen 170E may include a plurality of answer guides 187 respectively corresponding to a plurality of values that the driver information 125 can take. In this case, text of each answer guide 187 is determined according to the driver information 125 corresponding to a question. The answer guide 187 may be an operation portion that receives a touch operation, and in this case, the mobile terminal 1 can receive a touch operation on the answer guide 187 as input of an answer.

During display of the screen 170D or the screen 170E, in a case where an answer from the user U is input and the input answer is an appropriate answer, the mobile terminal 1 determines whether or not all the driver information 125 has been acquired in step S31 (FIG. 6) as described above.

In a case where there is unacquired driver information 125 (step S31: NO), the mobile terminal 1 returns to step S26, selects the driver information 125, and outputs a question in step S27. In this case, the mobile terminal 1 switches the display of the display 132 from the screen 170D or the screen 170E to the screen 170C.

In a case where all the driver information 125 has been acquired (step S31: YES), the mobile terminal 1 generates the assistance information 126 (step S17), switches the display of the display 132 to the screen 170F, and outputs the assistance information 126 (step S18).

The screen 170F is displayed as one form of output of the assistance information 126. The voice of the assistance information 126 output by the mobile terminal 1 is output during display of the screen 170F.

On the screen 170F, a vehicle object 181F, transition instruction portions 183 and 184, and an assistance information display portion 189 are disposed. The assistance information display portion 189 displays content of the assistance information 126. The assistance information display portion 189 displays a display object 150 and an attention object 160. The number of images or icons displayed on the assistance information display portion 189 is preferably four or less, and more preferably two or less as described above. In FIG. 7, as a preferable example, two images of the display object 150 and the attention object 160 are displayed on the assistance information display portion 189. Thus, the image displayed on the assistance information display portion 189 is likely to remain in memory for the user U who drives the vehicle M during the subsequent driving, and is likely to be recalled during the driving.

In the screen 170F, the vehicle object 181F may be an image imitating an expression corresponding to the weather indicated by the display object 150.

The attention object 160 is an image that functions as advice regarding driving of the vehicle M to the user U. The attention object 160 is, for example, an icon, a still image, or an animation indicating an object, a place, a person, or the like that the user U should pay attention to while driving the vehicle M. The attention object 160 is a display object indicating driving tricks or TIPS for the user U to drive the vehicle M. The attention object 160 is an image acquired by the assistance information generation unit 104 from the assistance DB 124 together with the display object 150 and included in the assistance information 126. For example, the attention object 160 is stored in the assistance DB 124 in association with the driver state classified by the state determination unit 103. Alternatively, the attention object 160 may be stored in the assistance DB 124 in association with a combination of values of the driver information 125. The state determination unit 103 may select one attention object 160 from among the plurality of attention objects 160 stored in the assistance DB 124 randomly or in accordance with the content of the display object 150 and cause the selected attention object 160 to be included in the assistance information 126. A message may be stored in the assistance DB 124 in association with each of the plurality of attention objects 160. In this case, the state determination unit 103 acquires a message corresponding to the attention object 160 from the assistance DB 124 and causes the message to be included in the assistance information 126.

During display of the screen 170F, a message output in a voice by the mobile terminal 1 includes a message corresponding to the display object 150. The voice of this message includes, for example, a wording “Today's driving forecast will be announced” in association with the display object 150 imitating the weather forecast. The voice of the message may include a wording explaining the content of the display object 150. The mobile terminal 1 utters, for example, “It will be cloudy and then rainy”. The voice of the message may include a wording explaining the content of the display object 150 in detail, and for example, the mobile terminal 1 may utter, “Since a slightly rough portion appears, there may be a case where normal driving ability cannot be exhibited.”. The mobile terminal 1 may output a message related to the attention object 160 as described above. For example, the mobile terminal 1 utters, in association with the attention object 160 that is an image of a bicycle, a wording such as “When passing a bicycle, if you slowly take a distance and drive, your wish may be fulfilled. Have a safe driving while imagining the feeling of the person riding the bicycle!”.

As described above, the mobile terminal 1 acquires the driver information 125 from biological information of the user U, information heard by using a question, and the like, and combines the plurality of pieces of driver information 125 to estimate a driver state indicating a state of the user U. The mobile terminal 1 specifies an ability of the user U that is insufficient or does not function well according to the driver state, and generates the assistance information 126 serving as an advice group that compensates for this. When generating the assistance information 126, the mobile terminal 1 selects appropriate advice according to the driver state of the user U. As a result, driving assistance can be provided to the user U who drives the vehicle M. By setting the information output from the mobile terminal 1 as a driving forecast which is a metaphor of a weather forecast, it is possible to cause the user U to understand that the information is only a prediction and to express the information such that the user U can intuitively easily understand the information.

### 2. Second Embodiment

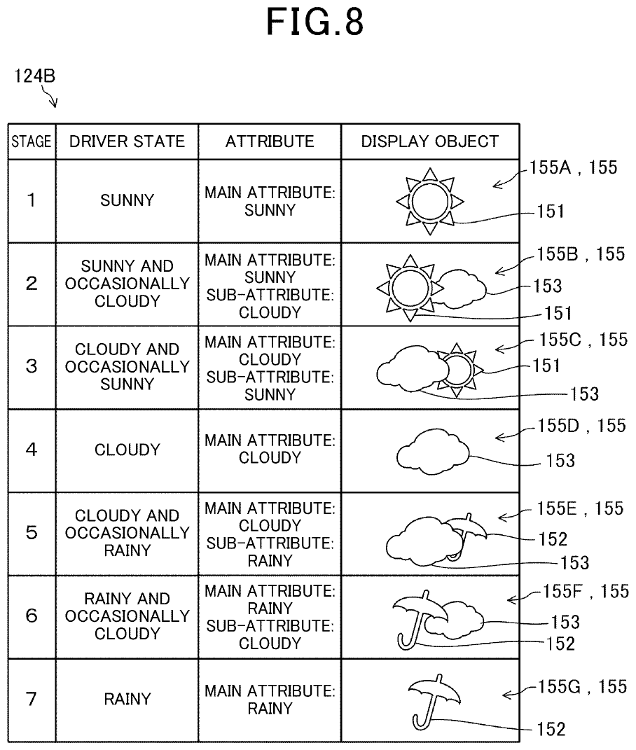

A second embodiment to which the present invention is applied will be described with reference to FIGS. 8 to 10. In the second embodiment described below, since configurations of the mobile terminal 1, WD 2, and the communication system 3 are common to those of the first embodiment, the same reference numerals are given to the configurations common to those of the first embodiment, and illustration and description thereof will be omitted.

### 2-1. Classification of Driver State

In the first embodiment, the example in which the mobile terminal 1 classifies a driver state of the user U into three stages of a positive state, a negative state, and an intermediate state has been described. In the second embodiment, the mobile terminal 1 classifies the driver state of the user U into seven stages.

FIG. 8 is a diagram illustrating a configuration example of display setting data 124B in the second embodiment.

The display setting data 124B is data stored in the assistance DB 124, and includes data corresponding to seven stages of classification of the driver state.

The display setting data 124A described in the first embodiment includes the display object 150 that reflects a temporal change in the driver state in association with a combination of the first driver state and the second driver state. In contrast, the display setting data 124B includes data regarding seven stages into which the driver state at the first time point and the driver state at the second time point are classified.

Specifically, the display setting data 124B associates seven stages with the classification of the driver state. The highest stage 1 of the seven stages indicates that a driver state is good, and a symbol of the driver state is “sunny”. The symbol of the driver state is a metaphor expressing the driver state, that is, the state of the user U, by imitating a weather forecast.

The lowest stage 7 of the seven stages indicates that a driver state is bad, and a symbol of the driver state is “rainy”. The stages between the stage 1 and the stage 7 are divided into the stage 2 to 6. The symbols of the driver state of each stage are: the stage 2 is “sunny and occasionally cloudy”, the stage 3 is “cloudy and occasionally sunny”, the stage 4 is “cloudy”, the stage 5 is “cloudy and occasionally rainy”, and the stage 6 is “rainy and occasionally cloudy”.

An attribute is set for the driver state at each stage. The attribute is a combination of a main attribute and a sub-attribute, the main attribute of the stages 1 and 2 is “sunny”, the main attribute of the stages 3 to 5 is “cloudy”, and the main attribute of the stages 6 and 7 is “rainy”. In other words, the display setting data 124B includes seven stages corresponding to a combination of three main attributes of sunny, cloudy, and rainy and a sub-attribute.

The display setting data 124B includes display objects 155 corresponding to the respective stages 1 to 7. A display object 155A corresponding to the stage 1 corresponds to “sunny” that is the driver state of the stage 1, and includes a first object 151 indicating “sunny”. A display object 155B corresponding to the stage 2 corresponds to “sunny and occasionally cloudy” that is the driver state of the stage 2, and includes the first object 151 and a third object 153. Similarly, display objects 155C to 155G are respectively associated with the stages 3 to 7. As described above, the display objects 155A to 155G are images expressing the driver state, that is, the state of the user U, by using images used in the weather forecast as metaphors.

The display object 155 includes a combination of an object indicating the main attribute and an object indicating the sub-attribute. For example, the display object 155F indicating the stage 5 is a combination of the second object 152 indicating the main attribute and the third object 153 indicating the sub-attribute.

The combination of the main attribute and the sub-attribute in the display setting data 124B does not express a temporal change in the driver state. For example, “sunny and occasionally cloudy” that is a symbol of the stage 2 expresses the driver state by adding a nuance of “cloudy” that is the sub-attribute to “sunny” that is the main attribute, and does not indicate that the driver state changes from “sunny” to “cloudy”. The same applies to the display object 155, and for example, the display object 155F indicating the stage 5 does not indicate that the driver state changes from “rainy” indicated by the second object 152 to “cloudy” indicated by the third object 153.

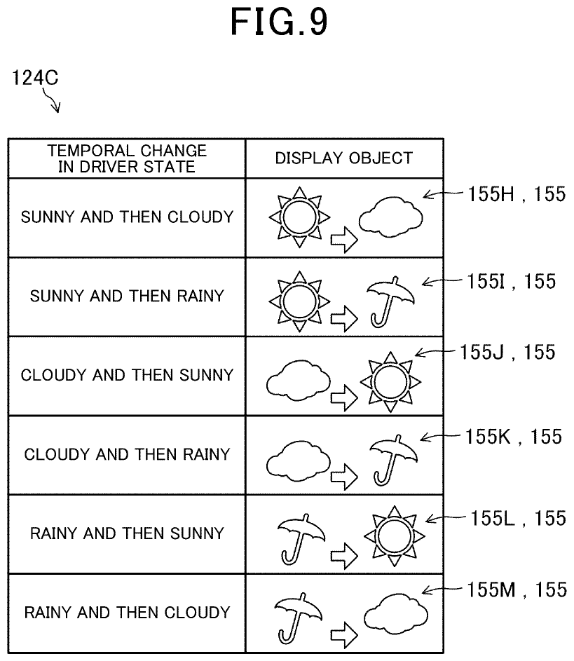

FIG. 9 is a diagram illustrating a configuration example of display setting data 124C in the second embodiment.

The display setting data 124C is data stored in the assistance DB 124, and includes data corresponding to six time-series changes in the driver state.

Specifically, the display setting data 124C includes six display objects 155 corresponding to six temporal changes of the main attribute of the driver state. For example, the display setting data 124C associates a display object 155I with a case where the main attribute of the driver state changes from “sunny” to “rainy”. The display object 155I includes the first object 151 and the second object 152. The display object 155I is an image expressing that the driver state changes from a good state to a bad state by using the first object 151 and the second object 152 and using a metaphor of a weather forecast. Similarly, display objects 155H, 155J, 155K, 155L, and 155M are images expressing time-series changes in the driver state by using metaphors of the weather forecast.

### 2-2. Operations of Mobile Terminal

The mobile terminal 1 uses the display setting data 124B to classify the driver state of the user U into seven stages in steps S13 and S14 in FIG. 5 according to the function of the state determination unit 103.

Specifically, in step S13, the mobile terminal 1 classifies the first driver state into seven stages on the basis of a result of analyzing the driver information 125 in step S12. In step S14, the mobile terminal 1 classifies the second driver state into seven stages on the basis of the result of analyzing the driver information 125 in step S12. Therefore, each of the first driver state and the second driver state is classified as one of the seven stages indicated by the display setting data 124B.

As a method in which the state determination unit 103 classifies the first driver state and the second driver state from the driver information 125, the method exemplified in the first embodiment may be used. For example, the state determination unit 103 may use a table in which a combination of values of the driver information 125A to 125D, a classification of the first driver state, and a classification of the second driver state are associated with each other. For example, the state determination unit 103 may determine each of the classification of the first driver state and the classification of the second driver state from the driver information 125 by using a function or a program. For example, the state determination unit 103 may determine each of the classification of the first driver state and the classification of the second driver state by calculating a value of the driver information 125. For example, the state determination unit 103 may determine each of the classification of the first driver state and the classification of the second driver state by using a learning model that associates the value of the driver information 125 with the driver state.

In step S15 in FIG. 5, the mobile terminal 1 acquires the display object 155 from the display setting data 124B or the display setting data 124C on the basis of the first driver state and the second driver state. In step S17, the mobile terminal 1 generates the assistance information 126 including the display object 155 acquired in step S15.

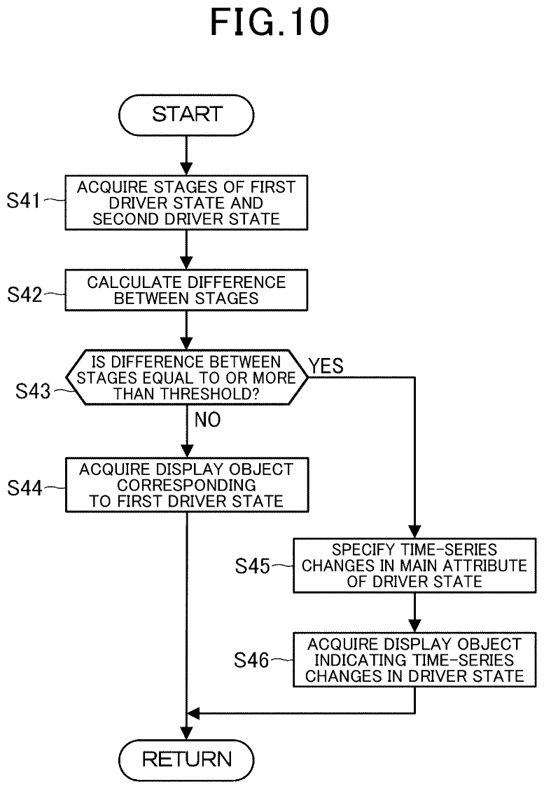

FIG. 10 is a flowchart illustrating an operation example of the mobile terminal 1 in the second embodiment, and illustrates an operation of acquiring the display object 155 in step S15 in FIG. 5. The operation in FIG. 10 is executed by the assistance information generation unit 104.

The mobile terminal 1 acquires a stage of the first driver state and a stage of the second driver state (step S41). The stage acquired in step S41 is a numerical value of each stage set in the display setting data 124B. The mobile terminal 1 calculates a difference between the stage of the first driver state and the stage of the second driver state (step S42), and determines whether the calculated difference is equal to or more than a threshold (step S43). The threshold is, for example, 3.

In a case where the difference between the stage of the first driver state and the stage of the second driver state is less than the threshold (step S43: NO), the mobile terminal 1 acquires the display object 155 associated with the stage of the first driver state from the display setting data 124B (step S44). That is, the mobile terminal 1 sets any one of the display objects 155A to 155G as a display object to be included in the assistance information 126.

In a case where the difference between the stage of the first driver state and the stage of the second driver state is equal to or more than the threshold (step S43: YES), the mobile terminal 1 specifies the time-series change in the main attribute on the basis of the main attribute of the first driver state and the main attribute of the second driver state (step S45). The mobile terminal 1 acquires the display object 155 corresponding to the change in the main attribute of the driver state specified in step S45 (step S46). That is, the mobile terminal 1 sets any one of the display objects 155H to 155M as a display object to be included in the assistance information 126.

After executing step S44 or step S46, the mobile terminal 1 proceeds to step S16 in FIG. 5.

As described above, in the second embodiment, the mobile terminal 1 classifies the driver state of the user U into seven stages. As a result, the mobile terminal 1 can more finely classify a state of the user U by using the driver information 125 obtained from an answer to a question to the user U.

The mobile terminal 1 classifies at least one of the first driver state at the first time point and the second driver state at the second time point into seven stages. As a result, the mobile terminal 1 can obtain a more detailed change in the state of the user U.

The mobile terminal 1 classifies each of the driver state at the first time point and the driver state at the second time point into seven stages. The mobile terminal 1 specifies a change in the driver state on the basis of the driver state at the first time point and the driver state at the second time point. The assistance information 126 displayed on the screen 170 by the mobile terminal 1 includes a driver state at the first time point or a display object corresponding to a change in the driver state. As a result, it is possible to finely classify the state of the user U and give detailed advice to the user U.

The mobile terminal 1 can determine whether or not the change in the state of the user U is large on the basis of the difference between the first driver state and the second driver state by using the threshold.

In a case where the change in the state of the user U is small, that is, in a case where the change in the stage of the driver state is smaller than the threshold, the mobile terminal 1 displays the display object 150 including any one of the display objects 155A to 155G indicating the first driver state on the screen 170. As a result, the user U is notified of the current state of the user U, and thus can be assisted in driving the vehicle M by calling attention to the driving.

In a case where the change in the state of the user U is large, that is, in a case where the change in the stage of the driver state is equal to or more than the threshold, the mobile terminal 1 displays the display object 150 including any one of the display objects 155H to 155M indicating the time-series change in the driver state on the screen 170. As a result, it is possible to notify the user U that there is a change in the state of the user U, and it is possible to effectively assist in the driving of the vehicle M by attracting more attention to the driving.

In any of the above cases, the mobile terminal 1 uses the image used for the weather forecast as a metaphor of the state of the user U. As a result, it is possible to notify of the state of the user U according to a method in which the state of the user U is easy to understand and is easily impressed. By using the metaphor of the weather forecast, it is possible to provide information regarding the state of the user U and assist in the driving of the vehicle M without causing the user U to feel tense or anxious.

### 3. Other Embodiments

Each of the above-described embodiments shows only one aspect, and can be freely modified and applied.

In the first embodiment, the example has been described in which the display object 150 corresponding to the combination of the first driver state and the second driver state is displayed on the screen 170. In the second embodiment, the example has been described in which the display object 155 corresponding to the first driver state or the display object 155 corresponding to the change in the main attribute of the driver state is displayed on the screen 170 on the basis of the difference between the first driver state and the second driver state. These are examples, and for example, the mobile terminal 1 may use only one of the first driver state and the second driver state for display. Specifically, the mobile terminal 1 may acquire and display the display object 150 or the display object 155 corresponding to either the first driver state or the second driver state on the screen 170.

In each of the above embodiments, an example has been described in which the assistance information generation unit 104 generates the assistance information 126 on the basis of both the classification of the first driver state at the first time point and the classification of the second driver state at the second time point. This is an example, and for example, the assistance information generation unit 104 may generate the assistance information 126 including the display object 155 corresponding to the first driver state on the basis of the result of classifying the first driver state at the first time point in the state determination unit 103. In this case, as the assistance information 126, the display object 155 expressing the first driver state is displayed on the screen 170. The assistance information generation unit 104 may cause advice or the like corresponding to the first driver state to be included in the assistance information 126. In these cases, the user U is notified of the state of the user U at the first time point by using an image or the like, and is notified of advice corresponding to the state. In this example, since the assistance information 126 is generated and output on the basis of the first driver state, there is an advantage that the information sent to the user U is simple and easily remembered in a more easily understandable manner. Since the processing of the assistance information generation unit 104 is simple, there is also an advantage that a processing load is light.

In each of the above embodiments, an example has been described in which in a case where the mobile terminal 1 has been able to acquire the driver information 125 from the WD 2, the mobile terminal 1 does not output a question corresponding to the acquired driver information 125. This is an example, and for example, the mobile terminal 1 may output a question about the driver information 125 of which a value has been specified on the basis of the information acquired from the WD 2. In this case, in a case where a value based on the information acquired from the WD 2 matches a value indicated by an answer of the user U to the question, the mobile terminal 1 may add additional information indicating that a reliability value is high to the value of the driver information 125. In a case where the value based on the information acquired from the WD 2 does not match the value indicated by the answer of the user U to the question, the mobile terminal 1 may add additional information indicating that the reliability value is low to the value of the driver information 125. Here, the mobile terminal 1 determines the value of the driver information 125 by giving priority to either the information obtained from the WD 2 or the answer of the user U.

As described above, the mobile terminal 1 may add additional information indicating the reliability to the value of the driver information 125. In this case, the mobile terminal 1 may change the message included in the assistance information 126 according to the additional information. For example, in a case where the driver information 125 with high reliability is obtained, the mobile terminal 1 causes a message including a definitive expression to be included in the assistance information 126. For example, in a case where the reliability of the driver information 125 is low, the mobile terminal 1 may generate the assistance information 126 including a message with a weakened expression.

FIGS. 4, 8, and 9 are diagrams schematically illustrating a configuration example of data stored in the assistance DB 124, and a configuration of each piece of data can be freely changed. For example, configurations, shapes, and the number of images of the display objects 150 and 155 included in the display setting data 124A, 124B, and 124C can be freely changed, and text may be included.

The vehicle M described in each of the above embodiments is an example of a moving object. The vehicle M is not limited to a four-wheeled vehicle, and may be a vehicle having five or more wheels or a vehicle having three or less wheels. For example, the vehicle M may be a motorcycle. The moving object may be a large vehicle such as a bus, a commercial vehicle, a work vehicle, or the like. The present disclosure is applicable to a moving object that is moved according to steering or driving of the user U. In addition to the above-described land moving object such as an automobile, the moving object may be a marine moving object such as a ship or a submersible, an aerial moving object such as an aircraft or an airship including an electric vertical take-off and landing aircraft (eVTOL), or a space moving object such as a spacecraft or an artificial satellite. The driving of the user U indicates that the user U operates and moves the moving object, and includes other operations such as steering.

In each of the above embodiments, an example has been described in which the operations illustrated in FIGS. 5 to 7 and 10 are executed by the portable mobile terminal 1 used by the user U, but these operations may be executed by, for example, a device installed in the vehicle M. Specifically, the present disclosure may be realized by a display audio (DA), a car navigation device, an in-vehicle infortainment (IVI) system, or other in-vehicle devices mounted on the vehicle M. The server 5 may execute some of the operations illustrated in FIGS. 5 to 7 and 10. For example, there may be a configuration in which the mobile terminal 1 executes communication with the WD 2, output of a question to the user U, reception of an answer input by the user U, and output of assistance information, and the server 5 executes other operations in steps S12 to S17 in FIG. 5. In this case, the mobile terminal 1, the server 5, and the WD 2 configure the communication system 3.

The processor 100 may be configured by a single processor or may be configured by a plurality of processors. The processor 100 may be hardware programmed to realize corresponding functional units. That is, the processor 100 may include, for example, an application specific integrated circuit (ASIC) or a field programmable gate array (FPGA).

The configuration of each unit illustrated in FIG. 2 is an example, and a specific implementation form is not particularly limited. That is, it is not always necessary to implement hardware corresponding to each unit individually, and the function of each unit may be realized by a single process executing a program. Some of the functions realized by software in the above-described embodiments may be realized by hardware, or some of the functions realized by hardware may be realized by software. In addition, the specific detailed configuration of each unit illustrated in FIG. 1 can be freely changed.

The step units of the operations illustrated in FIGS. 5, 6, and 10 are obtained by dividing the operations according to main processing content in order to facilitate understanding of the operation, and the present invention is not limited by the way or the name of division of the processing unit. The operations may be divided into a larger number of step units on the basis of the processing content. The operations may be divided into step units such that each of the step units includes a larger number of processes. The order of the steps may be changed as appropriate without departing from the concept of the present invention.

In a case where the driving assistance method for the mobile terminal 1 described above is realized by using the processor 100, a program executed by the processor 100 may be stored in a non-transitory recording medium, or may be configured in a mode of a transmission medium that transmits the program. For example, at least one of the control program 121 and the application 122 can be realized in a state of being recorded in a portable information recording medium. Examples of the information recording medium include a magnetic recording medium such as a hard disk, an optical recording medium such as a CD, and a semiconductor storage device such as a universal serial bus (USB) memory or a solid state drive (SSD), but other recording media may also be used.

### 4. Configurations Supported by Above Embodiments

The above embodiments support the following configurations.

(Configuration 1) A driving assistance device that outputs a plurality of questions for acquiring a plurality of pieces of driver information related to life of a driver of a moving object, acquires a plurality of pieces of driver information on the basis of answers of the driver to the plurality of respective questions, classifies a driver state related to at least one of a psychological state or a physical state of the driver on the basis of the plurality of pieces of driver information, generates assistance information including advice regarding driving on the basis of a classification result of the driver state, and outputs the assistance information.

According to the driving assistance device of Configuration 1, by outputting a question and causing the driver to input an answer, information regarding a state of the driver can be easily acquired, and advice corresponding to the state of the driver can be given. Consequently, it is possible to quickly classify the state of the driver and to assist the driver, which in turn contributes to the development of a sustainable transportation system.

(Configuration 2) The driving assistance device according to Configuration 1, further including a communication unit that executes communication with another device, in which, the communication unit attempts to acquire the driver information, and in a case where the communication unit has acquired any one or more of the plurality of pieces of the driver information, the question related to the driver information acquired by the communication unit is not output.

According to the driving assistance device of Configuration 2, it is possible to obtain information regarding the state of the driver according to a method different from a method in which the driver inputs an answer to a question.

(Configuration 3) The driving assistance device according to Configuration 1 or 2, in which the driver state is classified as any one of a positive state, a negative state, and an intermediate state between the positive state and the negative state.

According to the driving assistance device of Configuration 3, by classifying the driver state into three stages, it is possible to reduce a processing load required for generating assistance information in accordance with the state of the driver and to more quickly assist the driver.

(Configuration 4) The driving assistance device according to Configuration 1, in which the driver state is classified as any one of four or more stages including a positive state, a negative state, and an intermediate state.

According to the driving assistance device of Configuration 4, by classifying the driver state into four or more stages, assistance information finely corresponding to the state of the driver can be output to assist the driver.

(Configuration 5) The driving assistance device according to Configuration 1, in which, in a process of classifying the driver state, the driver state at a first time point is classified, and the assistance information including a classification result of the driver state at the first time point is generated.

According to the driving assistance device of Configuration 5, assistance information including a classification result of the driver state at the first time point is output. Therefore, the content of the information output to the driver can be simplified, a load of understanding and memory of the driver can be reduced, and a processing load for generating the assistance information can be reduced.

(Configuration 6) The driving assistance device according to Configuration 5, in which the assistance information including a display object corresponding to the classification result of the driver state at the first time point is generated as the assistance information, and the assistance information is output by displaying the display object on a display unit.

According to the driving assistance device of Configuration 6, by displaying the display object expressing the classification result of the driver state at the first time point, information corresponding to the state of the driver can be output to the driver through display. Therefore, a load on the driver to understand and remember the output assistance information can be further reduced.

(Configuration 7) The driving assistance device according to any one of Configuration 1 to Configuration 4, in which, in a process of classifying the driver state, the driver state at a first time point is classified, the driver state at a second time point after a predetermined time has elapsed from the first time point is predicted and classified, and the assistance information including a classification result of the driver state at the first time point and a classification result of the driver state at the second time point is generated.

According to the driving assistance device of Configuration 7, by classifying a state of the driver and a temporal change in the state of the driver, it is possible to give advice corresponding to the change in the state of the driver.

(Configuration 8) The driving assistance device according to Configuration 7, in which the assistance information including a display object corresponding to the classification result of the driver state at the first time point and the classification result of the driver state at the second time point is generated as the assistance information, and the assistance information is output by displaying the display object on a display unit.

According to the driving assistance device of Configuration 8, since the display object corresponding to the state of the driver and the temporal change in the state of the driver is displayed, it is possible to give advice to the driver in an easily understandable manner.

(Configuration 9) The driving assistance device according to Configuration 8, in which, in the process of classifying the driver state, the driver state at the first time point is classified as one of a positive state, a negative state, and an intermediate state between the positive state and the negative state, the driver state at the second time point is classified as one of the positive state, the negative state, and the intermediate state, and the display object included in the assistance information expresses a classification result of the driver state at the first time point and a classification result of the driver state at the second time point while reflecting a temporal transition, by using at least one of a first object indicating the positive state, a second object indicating the negative state, and a third object indicating the intermediate state.

According to the driving assistance device of Configuration 9, by expressing the state of the driver and the change in the state as the display object reflecting the temporal transition, it is possible to give advice in accordance with the state of the driver in a mode in which the display object is easy for the driver to visually recognize.

(Configuration 10) The driving assistance device according to Configuration 9, in which the display object has a configuration imitating a weather forecast, the first object is an image imitating an expression of sunny weather, the second object is an image imitating an expression of rainy weather, and the third object is an image imitating an expression of cloudy weather.

According to the driving assistance device of Configuration 10, advice reflecting the state of the driver is given by using the image imitating the weather forecast. Therefore, it is possible to give advice that is easy to understand to easily remain in the memory of the driver, and to more appropriately assist the driver.

(Configuration 11) The driving assistance device according to Configuration 8, in which, in the process of classifying the driver state, at least one of the driver state at the first time point and the driver state at the second time point is classified into seven stages.

According to the driving assistance device of Configuration 11, by finely classifying the state of the driver, assistance can be performed in more detail according to the state of the driver.

(Configuration 12) The driving assistance device according to Configuration 11, in which, in the process of classifying the driver state, each of the driver state at the first time point and the driver state at the second time point is classified into seven stages, and a change in the driver state is specified on the basis of the driver state at the first time point and the driver state at the second time point, and the assistance information includes the display object corresponding to the driver state at the first time point or the change in the driver state.

According to the driving assistance device of Configuration 12, the state of the driver can be finely classified, and it is possible to assist in driving in accordance with the state of the driver or the change in the state of the driver.

(Configuration 13) The driving assistance device according to any one of Configuration 1 to Configuration 12, in which the assistance information includes information indicating driving tricks or TIPS that the driver should keep in mind in driving the moving object.

According to the driving assistance device of Configuration 13, the driving tricks or TIPS can be transmitted to the driver.

(Configuration 14) The driving assistance device according to Configuration 13, in which the assistance information includes an assistance display object expressing the driving tricks or TIPS.

According to the driving assistance device of Configuration 14, the driving tricks or TIPS can be transmitted to the driver in an easily understandable manner to easily remain in the memory of the driver.

(Configuration 15) A driving assistance method of causing a driving assistance device including a processor to execute: outputting a plurality of questions for acquiring a plurality of pieces of driver information related to life of a driver of a moving object; acquiring the plurality of pieces of driver information on the basis of answers of the driver to the plurality of respective questions; classifying a driver state related to at least one of a psychological state or a physical state of the driver on the basis of the plurality of pieces of driver information; generating assistance information including advice regarding driving on the basis of a classification result of the driver state; and outputting the assistance information.

According to the driving assistance method of Configuration 15, by outputting a question and causing the driver to input an answer, information regarding a state of the driver can be easily acquired, and advice corresponding to the state of the driver can be given. Consequently, it is possible to quickly classify the state of the driver and to assist the driver, which in turn contributes to the development of a sustainable transportation system.

(Configuration 16) A program executable by a computer, the program causing the computer to execute: outputting a plurality of questions for acquiring a plurality of pieces of driver information related to life of a driver of a moving object; acquiring a plurality of pieces of driver information on the basis of answers of the driver to the plurality of respective questions; classifying a driver state related to at least one of a psychological state or a physical state of the driver on the basis of the plurality of pieces of driver information; generating assistance information including advice regarding driving on the basis of a classification result of the driver state; and outputting the assistance information.

According to the program of Configuration 16, by outputting a question and causing the driver to input an answer, information regarding a state of the driver can be easily acquired, and advice corresponding to the state of the driver can be given. Consequently, it is possible to quickly classify the state of the driver and to assist the driver, which in turn contributes to the development of a sustainable transportation system.

### REFERENCE SIGNS LIST

1 Mobile terminal (driving assistance device)
2 Wearable device (another device)
3 Communication system
5 Server
10 Control unit
100 Processor
101 Acquisition unit
102 Reception unit
103 State determination unit
104 Assistance information generation unit
105 Output control unit
120 Memory
121 Control program
122 Application (program)
123 Setting data
124 Assistance database
124A, 124B, 124C Display setting data
125, 125A, 125B, 125C, 125D Driver information
126 Assistance information
130 First communication unit (communication unit)
131 Second communication unit
132 Display (display unit, output unit)
133 Touch sensor
134 Microphone
135 Speaker (output unit)
150, 150A to 150D Display object
151 First object
152 Second object
153 Third object
155, 155A to 155M Display object
170, 170A to 170F Screen
181, 181A to 181F Vehicle object
182 Start instruction portion
183, 184 Transition instruction portion
185 Busy display
186 Utterance guide
187 Answer guide
M Vehicle (moving object)
NW Communication network
U User (driver)

What is claimed is:

1. A driving assistance device that executes:
outputting a plurality of questions for acquiring a plurality of pieces of driver information related to a life of a driver of a moving object;
acquiring the plurality of pieces of driver information on the basis of answers of the driver to the plurality of respective questions;
classifying a driver state related to at least one of a psychological state and a physical state of the driver on the basis of the plurality of pieces of driver information;
generating assistance information including advice regarding driving on the basis of a classification result of the driver state; and
outputting the assistance information.

2. The driving assistance device according to claim 1, further comprising
a communication unit that executes communication with another device, wherein
the communication unit attempts to acquire the driver information, and
in a case where the communication unit has acquired any one or more of the plurality of pieces of driver information, the question related to the driver information acquired by the communication unit is not output.

3. The driving assistance device according to claim 1, wherein the driver state is classified as one of a positive state, a negative state, and an intermediate state between the positive state and the negative state.

4. The driving assistance device according to claim 1, wherein the driver state is classified as one of four or more stages including a positive state, a negative state, and an intermediate state.

5. The driving assistance device according to claim 1, wherein,
in a process of classifying the driver state, the driver state at a first time point is classified, and
the assistance information including a classification result of the driver state at the first time point is generated.

6. The driving assistance device according to claim 5, wherein
the assistance information including a display object corresponding to the classification result of the driver state at the first time point is generated as the assistance information, and
the assistance information is output by displaying the display object on a display unit.

7. The driving assistance device according to claim 1, wherein
in a process of classifying the driver state, the driver state at a first time point is classified, the driver state at a second time point after a predetermined time has elapsed from the first time point is predicted and classified, and
the assistance information including a classification result of the driver state at the first time point and a classification result of the driver state at the second time point is generated.

8. The driving assistance device according to claim 7, wherein
the assistance information including a display object corresponding to the classification result of the driver state at the first time point and the classification result of the driver state at the second time point is generated as the assistance information, and
the assistance information is output by displaying the display object on a display unit.

9. The driving assistance device according to claim 8, wherein
in the process of classifying the driver state, the driver state at the first time point is classified as one of a positive state, a negative state, and an intermediate state between the positive state and the negative state, the driver state at the second time point is classified as one of the positive state, the negative state, and the intermediate state, and
the display object included in the assistance information expresses a classification result of the driver state at the first time point and a classification result of the driver state at the second time point while reflecting a temporal transition, by using at least one of a first object indicating the positive state, a second object indicating the negative state, and a third object indicating the intermediate state.

10. The driving assistance device according to claim 9, wherein the display object has a configuration imitating a weather forecast, the first object is an image imitating an expression of sunny weather, the second object is an image imitating an expression of rainy weather, and the third object is an image imitating an expression of cloudy weather.

11. The driving assistance device according to claim 8, wherein, in the process of classifying the driver state, at least one of the driver state at the first time point and the driver state at the second time point is classified into seven stages.

12. The driving assistance device according to claim 11, wherein
in the process of classifying the driver state, each of the driver state at the first time point and the driver state at the second time point is classified into seven stages, and a change in the driver state is specified on the basis of the driver state at the first time point and the driver state at the second time point, and
the assistance information includes the display object corresponding to the driver state at the first time point or the change in the driver state.

13. The driving assistance device according to claim 1, wherein the assistance information includes information indicating driving tricks or TIPS that the driver is to keep in mind in driving the moving object.

14. The driving assistance device according to claim 13, wherein the assistance information includes an assistance display object expressing the driving tricks or TIPS.

15. A driving assistance method of causing a driving assistance device including a processor to execute:
outputting a plurality of questions for acquiring a plurality of pieces of driver information related to a life of a driver of a moving object;
acquiring the plurality of pieces of driver information on the basis of answers of the driver to the plurality of respective questions;
classifying a driver state related to at least one of a psychological state and a physical state of the driver on the basis of the plurality of pieces of driver information;
generating assistance information including advice regarding driving on the basis of a classification result of the driver state; and
outputting the assistance information.

16. A non-transitory computer-readable storage medium storing a program executable by a computer, the program causing the computer to execute:
outputting a plurality of questions for acquiring a plurality of pieces of driver information related to life of a driver of a moving object;
acquiring the plurality of pieces of driver information on the basis of answers of the driver to the plurality of respective questions;
classifying a driver state related to at least one of a psychological state or a physical state of the driver on the basis of the plurality of pieces of driver information;
generating assistance information including advice regarding driving on the basis of a classification result of the driver state; and
outputting the assistance information.
